(19) United States
(12) Patent Application Publication
(10) Pub. No.: US 2008/0082004 A1
(43) Pub. Date: Apr. 3, 2008

(54) BLOOD PRESSURE MONITOR

(75) Inventors: Matthew John Banet, Del Mar, CA (US); Michael James Thompson, San Diego, CA (US); Zhou Zhou, La Jolla, CA (US)

Correspondence Address: Triage Wireless, Inc.;Matthew John Banet, 6540 LUSK BLVD., C200, SAN DIEGO, CA, 92121, US

(73) Assignee: TRIAGE WIRELESS, INC., San Diego, CA (US)

(21) Appl. No.: 11/530,076

(22) Filed: Sep. 8, 2006

(51) Int. Cl. A61B 5/02

(52) U.S. Cl. 600/485

(57) ABSTRACT

The invention provides a method for measuring a blood pressure value of a user featuring the following steps: 1) generating optical, electrical, and acoustic waveforms with, respectively, optical, electrical, and acoustic sensors attached to a single substrate that contacts a user; 2) determining at least one parameter by analyzing the optical and acoustic waveforms; and 3) processing the parameter to determine the blood pressure value for the user.

### CROSS REFERENCES TO RELATED APPLICATION

Not Applicable

### STATEMENT REGARDING FEDERALLY SPONSORED RESEARCH OR DEVELOPMENT

Not Applicable

### BACKGROUND OF THE INVENTION

1. Field of the Invention

The present invention relates to medical devices for monitoring vital signs such as blood pressure.

2. Description of the Related Art

Pulse transit time (‘PTT’), defined as the transit time for a pressure pulse launched by a heartbeat in a patient's arterial system, has been shown in a number of studies to correlate to both systolic and diastolic blood pressure. In these studies PTT is typically measured with a conventional vital signs monitor that includes separate modules to determine both an electrocardiogram (ECG) and pulse oximetry. During a conventional PTT measurement, multiple electrodes typically attach to a patient's chest to determine a time-dependent ECG characterized by a sharp spike called the ‘QRS complex’. This feature indicates an initial depolarization of ventricles within the heart and, informally, marks the beginning of the heartbeat. Pulse oximetry is typically measured with a clothespin-shaped device that clips to the patient's index finger, and includes optical systems operating in both the red and infrared spectral regions. In addition to measuring a pulse oximetry value, this method yields a time-dependent waveform, called a plethysmograph. The plethysmograph indicates both heart rate and a volumetric change in an underlying artery in the finger caused by the propagating pressure pulse.

In many studies PTT is calculated from the time separating the onset of the QRS complex to the foot of the plethysmograph. Alternatively, PTT can be calculated as the time separating signals measured by two sensors (e.g. optical or pressure sensors), each sensitive to the propagating pressure pulse, placed at different locations on the patient's body. In both cases, PTT depends primarily on arterial resistance, arterial compliance, the propagation distance (closely approximated by the patient's arm length), and of course blood pressure. Typically a high blood pressure results in a shorter PTT.

A number of issued U.S. Patents describe the relationship between PTT and blood pressure. For example, among others, U.S. Pat. Nos. 5,316,008; 5,857,975; 5,865,755; and 5,649,543 each teach an apparatus that includes conventional sensors that measure an ECG and plethysmograph that are processed to measure PTT. U.S. Pat. Nos. 6,511,436; 6,599,251; and 6,723,054 each teach an apparatus that includes a pair of optical or pressure sensors, each sensitive to a propagating pressure pulse, that measure PTT. As described in these patents, a microprocessor associated with the apparatus processes the PTT value to estimate blood pressure.

PTT-based measurements of blood pressure are complicated by a number of factors, one of which is the many time-dependent processes associated with each heartbeat that may correlate in a different way with blood pressure, or in fact may not correlate at all. For example, prior to the initial depolarization of the ventricles (marked by the QRS complex), the mitral valve opens and lets blood flow from the left atrium into the left ventricle. This causes the ventricle to fill with blood and increase in pressure. After the onset of the QRS, the mitral valve closes and the aortic valve opens. When the heart contracts, blood ejects into the aorta until the aortic valve closes. The time separating the onset of the QRS and the opening of the aortic valve is typically called the pre-injection period, or ‘PEP’. The time separating opening and closing of the aortic valve is called the left ventricular ejection period, or ‘LVET’. LVET and PEP, along with additional time-dependent properties associated with each heartbeat, are typically included in a grouping of properties called systolic time intervals, or ‘STIs’.

PTT and LVET can be measured with a number of different techniques, such as impedance cardiography (‘ICG’) and by measuring a time-dependent acoustic waveform, called a phonocardiogram (‘PCG’), with an acoustic sensor. The PCG, characterized by acoustic signatures indicating the closing (and not opening) of the mitral and aortic valves, is typically coupled with an ECG to estimate PEP and LVET. For example, U.S. Pat. Nos. 4,094,308 and 4,289,141 each teach an apparatus that measures a PCG and ECG, and from these waveforms estimates PEP and LVET. U.S. Pat. No. 7,029,447 teaches an apparatus using transit times calculated from an ICG measurement to determine blood pressure.

Studies have also shown that a property called vascular transit time (‘VTT’), measured from a first feature in a PCG and a plethysmograph measured from a patient's finger, can correlate to blood pressure. Such a study, for example, is described in an article entitled ‘Evaluation of blood pressure changes using vascular transit time’, Physiol. Meas. 27, 685-694 (2006). In addition, studies have shown that PEP and LVET, taken alone, can correlate to blood pressure. These studies typically require multiple sensors placed on the patient's body to measure time-dependent waveforms that are processed to determine PEP and LVET. Studies that relate these properties to blood pressure, for example, are described in ‘Systolic Time Intervals in Man’, Circulation 37, 149-159 (1968); ‘Relationship Between Systolic Time Intervals and Arterial Blood Pressure’, Clin. Cardiol. 9, 545-549 (1986); ‘Short-term variability of pulse pressure and systolic and diastolic time in heart transplant recipients’, Am. J. Physiol. Heart Circ. Physiol. 279, H122-H129 (2000); and ‘Pulse transit time measured from the ECG: an unreliable marker of beat-to-beat blood pressure’, J. App. J. Physiol. 100, 136-141 (2006).

### SUMMARY OF THE INVENTION

To address any deficiencies in the prior art, the present invention provides blood pressure monitor featuring a substrate that includes small-scale optical, electrical, and acoustic sensors. The substrate, for example, may be a single, continuous component, or alternatively may consist of a first component (e.g. a disposable adhesive material) that connects or attaches to a second component (e.g. a non-disposable insert that comprises the sensors). The sensors measure, respectively, time-dependent optical, electrical and acoustic waveforms that a processor then analyzes as described in detail below to determine blood pressure. In this way, the substrate replaces a conventional cuff to make a rapid, comfortable measurement of blood pressure.

The substrate can be integrated into a number of product configurations. For example, it can be attached to an adhesive backing and used as a stand-alone patch. In this configuration, a cable with a detachable connector typically connects to a tab connector fabricated directly onto the substrate. The other end of the cable connects to a hand-held console. This way, the patch can be adhered to a patient for an extended period of time to make quasi-continuous measurements, or can simply be connected to the console to make sporadic, one-time measurements. In another embodiment, the optical, electrical, and acoustic sensors are included in a circuit board attached to the end of the cable. In this case the circuit board can ‘snap’ into a disposable adhesive sensor, which in turn attaches to the patient. By including the relatively expensive electrical components in the cable, this embodiment minimizes the cost of the disposable component, which is comprised mostly of an adhesive pad and solid, conductive gel associated with the electrodes.

In other embodiments, the substrate can attach to a flexible backing (composed of, e.g., rubber, fiberglass, plastic, or similar flexible materials) and connect to a cable that is permanently attached to the console. Here, to make a blood pressure measurement, a user holds the substrate to their chest with one hand, and with the other hand holds the console and monitors the measurement. In yet another embodiment, the substrate connects directly to a back surface of the console, which is then held to the chest to make a blood pressure measurement.

A discovery that makes the above-described product configurations possible is that when the substrate is held to a specific area of a patient's chest, typically located a few centimeters below their ‘sternal’ notch, it can simultaneously measure optical, electrical, and acoustic waveforms with no external sensors. This is possible because: 1) the proximity of this area to the heart allows the acoustic sensor to measure the acoustic waveform; 2) an abundance of capillaries, which means the optical waveform can be measured in a reflective mode; and 3) the strong electrical activity of the heart in this area, meaning the electrical waveform can be measured with a high signal-to-noise ratio even when the electrodes are relatively close together.

Specifically, in one aspect, the invention provides a method for measuring a blood pressure value that features the following steps: 1) generating optical, electrical, and acoustic waveforms with, respectively, optical, electrical, and acoustic sensors attached to a single substrate; 2) determining at least one parameter by analyzing the optical and acoustic waveforms; and 3) processing the parameter to determine a blood pressure value.

In embodiments, to generate the optical waveform, the optical sensor irradiates a first region (e.g. an area below the sternal notch) with a light source (e.g. an LED), and then detects radiation reflected from this region with a photodetector. The signal from the photodetector passes to an analog-to-digital converter, where it is digitized so that it can be analyzed with a processor. The analog-to-digital converter can be integrated directly into the processor, or can be a stand-alone circuit component. Typically the radiation from the light source has a wavelength in a ‘green’ spectral region, typically between 520 and 590 nm. Alternatively, the radiation can have a wavelength in the infrared spectral region, typically between 800 and 1100 nm. To detect this radiation, the optical sensor includes a light detector, e.g. a photodiode or phototransistor. In preferred embodiments the light source and the light detector are included in the same housing or electronic package.

To generate the electrical waveform, the electrical sensor detects first and second electrical signals with, respectively, first and second electrodes. The electrical signals are then processed (e.g. with a multi-stage differential amplifier and band-pass filters) to generate a time-dependent electrical waveform similar to an ECG. The electrical sensor typically includes a third electrode, which generates a ground signal or external signal that is further processed to, e.g., reduce noise-related artifacts in the electrical waveform. In embodiments, the electrodes are disposed on opposite ends of the substrate, and are typically separated by a distance of at least two inches. In other embodiments, the electrodes include an Ag/AgCl material (e.g., an Ag/AgCl paste sintered to a metal contact) and a conductive gel. Typically a first surface of the conductive gel contacts the Ag/AgCl material, while a second surface is covered with a protective layer. The protective layer prevents the gel from drying out when not in use, and typically has a shelf life of about 24 months. In still other embodiments, the electrodes are made from a conductive material such as conductive rubber, conductive foam, conductive fabric, and metal.

To generate the acoustic waveform, the acoustic sensor typically includes a microphone or piezoelectric device that measures low-frequency pressure waves (e.g. sounds) from the user's heart. This results in a time-dependent acoustic waveform that typically includes two ‘packets’ comprised of frequency components typically ranging from 40-500 Hz. The packets correspond to closing of the mitral and aortic valves. The acoustic sensor can also contact a non-conductive impedance-matching gel, such as Vaseline®, to decrease acoustic reflections at the skin/sensor interface. This typically increases the magnitude of the measured acoustic waveform.

During a measurement, the processor analyzes the various waveforms to determine one or more time-dependent parameters, e.g. VTT, PTT, PEP, or LVET, which are then further processed to determine blood pressure. The processor can further process a waveform, e.g. take a second derivative or ‘fit’ the rise or fall times of the optical waveform with a mathematical function, to determine additional properties relating to blood pressure. For example, in one embodiment, the microprocessor determines at least one parameter by analyzing a first point from a pulse within the optical waveform and a second point from a feature representing a heart sound within the acoustic waveform (to estimate VTT). In another embodiment, the processor determines a second parameter by analyzing a point from a QRS complex within the electrical waveform and a point from either a pulse within the optical waveform (to estimate PTT) or a point within the acoustic waveform (to estimate PEP). In yet another embodiment, the processor analyzes points representing two heart sounds from the acoustic waveform (to estimate LVET).

Once these parameters are determined, the processor analyzes them with a mathematical model to determine the user's blood pressure. For example, the processor can process one or more parameters with a linear model, characterized by a slope and a y-intercept, to relate it (or them) to a blood pressure value. Alternatively, the processor can relate one or more parameters to blood pressure using a relatively complex model, such as one that includes a polynomial, exponential, or a non-linear set of equations. Once the various parameters are related to blood pressure, several ‘sub-values’ can be determined and concatenated into a single blood pressure value using, e.g., a pre-determined weighted average. The above-mentioned models can also use calibration values, e.g. calibration values from a cuff-based system or arterial line, to increase the accuracy of the blood pressure calculation.

In another aspect, the invention provides a monitor for measuring a user's blood pressure featuring a substrate that includes the above-described optical, electrical, and acoustic sensors. The substrate, for example, can be incorporated into disposable patch or a hand-held pad, both of which contact an area below the sternal notch region to make a blood pressure measurement. In embodiments, the substrate is a thin printed circuit board with metallized traces. Alternatively, the substrate can be a flexible material, such as a plastic band, with traces made from a conductive ink or epoxy.

In another aspect, the substrate features the optical, electrical, and acoustic sensors and connects to a hand-held console that includes a display, processor, and non-volatile memory. Here, the two components can connect using a flexible cable that includes an adjustable clip configured to attach and detach to electrical leads adhered directly to the substrate's surface. In this embodiment, the substrate may be enclosed in a flexible backing made from a material such rubber, plastic, foam, cloth, fiberglass, composite materials, or leather. The flexible backing may additionally include a handle to make it easier to position on the patient.

In another embodiment, the substrate attaches directly to the back of the console so that the optical, electrical, and acoustic sensors are on one side, and the display is on the other. In this case the user holds the monitor up to their chest to make a blood pressure measurement.

The processor within the monitor runs compiled computer code that executes an algorithm described in detail below, along with a graphical user interface that renders on the display. The display typically includes a touchscreen and the console a touchscreen controller. In this case, the graphical user interface typically includes multiple icons configured so that, when contacted through the touchscreen, activate a function on the processor. In other embodiments, the processor communicates with an Internet-accessible website through a serial port (e.g. a USB port) or a wireless interface (e.g. a modem operating a Bluetooth®, WiFi®, WiMax®, Zigbee®, CDMA, GSM, or comparable protocol).

The invention has a number of advantages. In general, because it lacks a cuff, the monitor according to the invention measures blood pressure in a simple, rapid, pain-free manner. Measurements can be made throughout the day with little or no inconvenience to the user. Moreover, the optical, electrical, and acoustic sensors are integrated on a single substrate connected to a console with a single wire. This means vital signs and related waveforms, such as blood pressure, heart rate, ECG, optical plethysmograph, and respiration rate, can be measured with a minimal amount of wires and patches connected to the patient. This can make the patient more comfortable, particularly in a hospital setting. The monitor can be used for one-time, quasi-continuous, or ambulatory measurements, meaning the patient can be well characterized both in and out of the hospital.

These and other advantages are described in detail in the following description, and in the claims.

### BRIEF DESCRIPTION OF THE DRAWINGS

FIG. 1 shows a front view of a blood pressure monitor according to the invention that features a console connected by a cable to a flexible foam pad that includes the substrate;

FIG. 2 shows a schematic view of the blood pressure monitor of FIG. 1 measuring a patient underneath their sternal notch;

FIG. 3A shows a graph of time-dependent electrical, optical, and acoustic waveforms measured with the blood pressure monitor of FIG. 1;

FIG. 3B shows a graph of the time-dependent electrical, optical, and acoustic waveforms shown in FIG. 3A plotted over a relatively short time scale;

FIG. 4 shows an equation used by an algorithm running on a microprocessor within the blood pressure monitor of FIG. 1 to calculate blood pressure;

FIG. 5 shows a graph of VTT* plotted as a function of systolic blood pressure, along with a linear fit to these data, for a single patient;

FIGS. 6A and 6B show, respectively, front and back views of a blood pressure monitor according to the invention wherein the flexible foam pad that includes the substrate connects directly to the console; and,

FIG. 7 is a front view of a disposable, adhesive patch that includes the substrate and connects through a cable to a blood pressure monitor according to the invention.

### DETAILED DESCRIPTION OF THE INVENTION

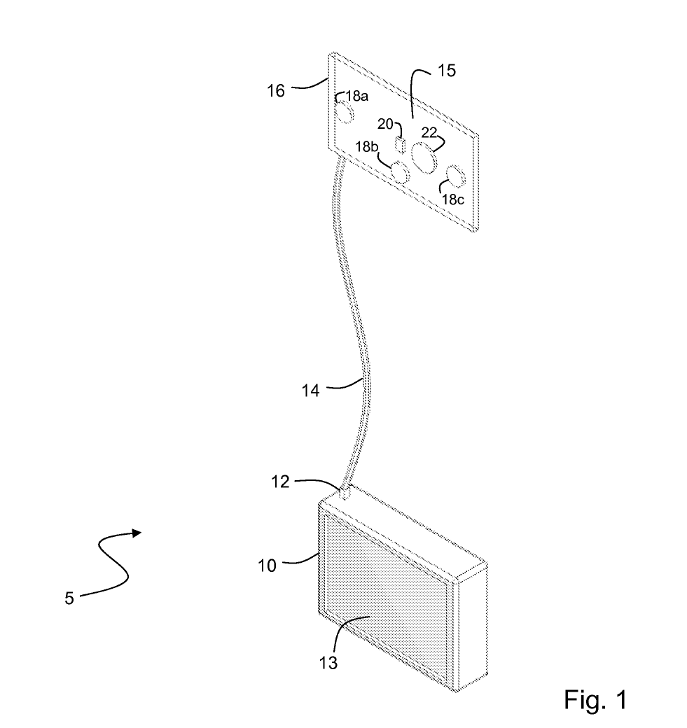

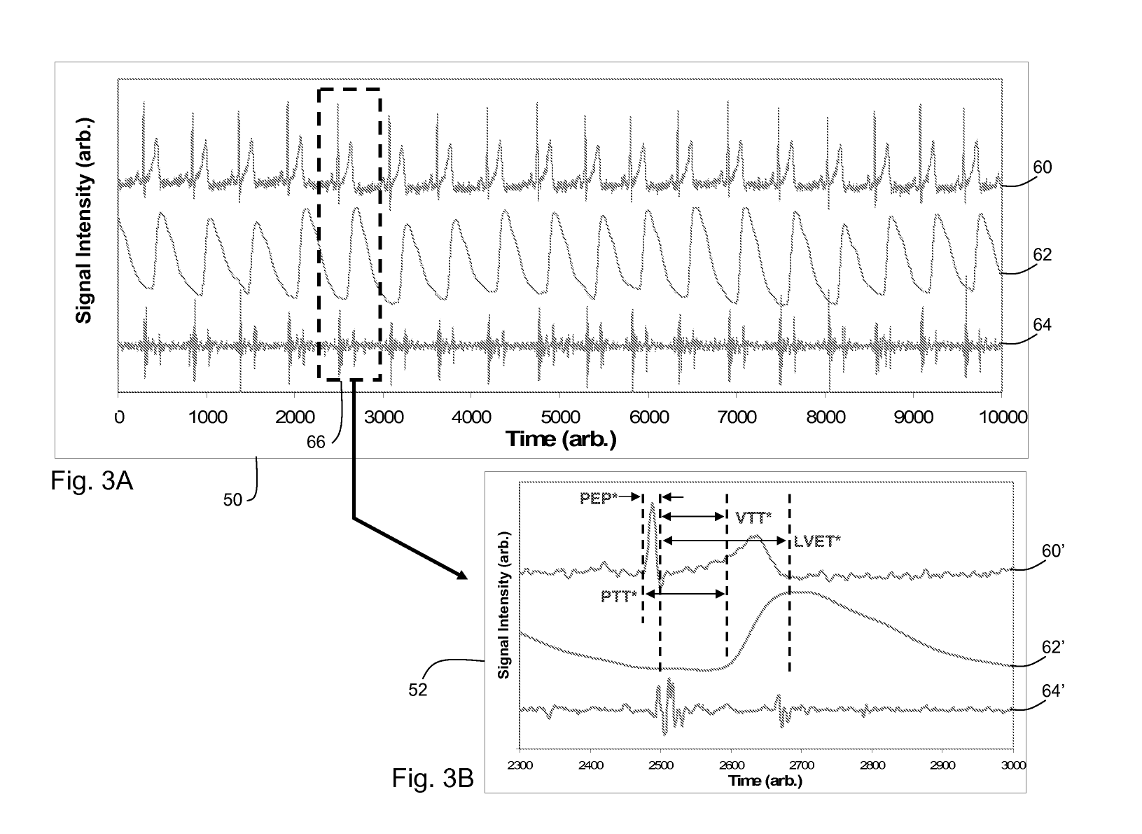

FIG. 1 shows a preferred embodiment of a blood pressure monitor 5 according to the invention that includes a console 10 that attaches to a flexible foam pad 16 through a cable 14. The flexible foam pad 16 includes a substrate 15 that supports sensors that measure time-dependent electrical, optical, and acoustic waveforms (shown in FIGS. 3A, 3B). The substrate 15 is preferably a flexible printed circuit board that adheres to the foam pad 16. During operation, the flexible foam pad 16 preferably contacts an area below a patient's sternal notch to measure the various waveforms from a patient. A microprocessor in the console 10 analyzes the waveforms to determine the systolic time intervals, which are then processed with an algorithm and a weighted average to determine the patient's real-time blood pressure.

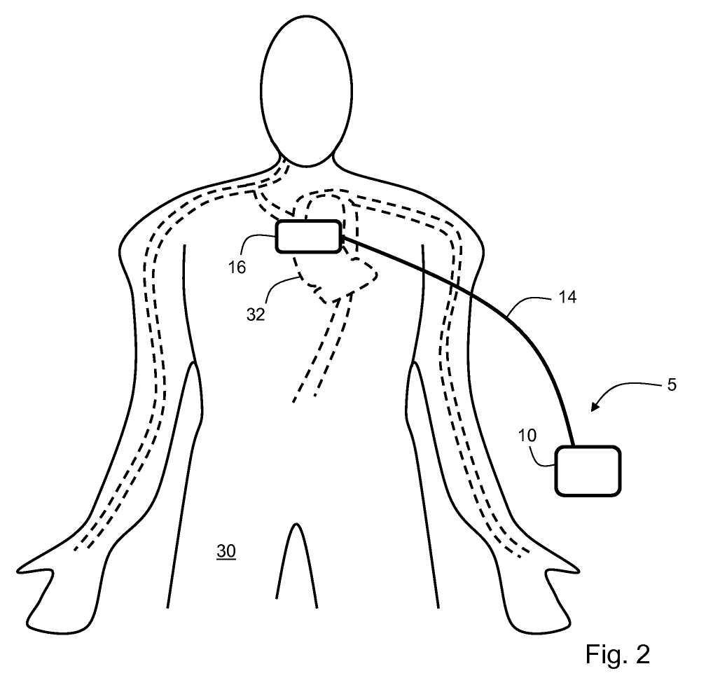

The flexible foam pad 16 preferably includes three electrodes 18a-c that measure two electrical signals and a ground (or other) signal from the patient. Two of the electrodes 18a, 18c are preferably spaced apart by at least two inches so that, when the flexible foam pad 16 contacts the patient (as shown in FIG. 2), the two electrodes 18a and 18c measure signals that can be processed with a differential amplifier and band-pass filters to determine an ECG-like electrical waveform. The flexible foam pad 16 additionally includes a reflective optical sensor 20 that includes a photodetector and a light-emitting diode (LED) that typically emits green radiation (λ=520-590 nm) to measure a reflective optical waveform representing blood flowing in underlying capillaries. A preferred optical sensor 30 (manufacturer: TAOS, Inc.; part number: TRS1755) includes a green LED light source (567 nm wavelength) and a light-to-voltage converter in a common housing. The flexible foam pad 16 additionally includes a piezoelectric acoustic sensor 22 that detects sounds waves following each of the patient's heartbeat to generate an acoustic waveform. The preferred piezoelectric acoustic sensor is preferably a Condenser Microphone Cartridge (manufacturer: Panasonic; part number: WM-55D103) that detects sounds waves following each of the patient's heartbeats to generate an acoustic waveform, also called a phonocardiogram.

Referring to FIG. 2, during operation the blood pressure monitor 5 is operated so that the console 10 is held in one hand of the patient 30 while the other hand holds the flexible foam pad 16 to the chest. In this way, the pad 16 is proximal to the patient's heart 32, a location that allows it to simultaneously measure optical, electrical, and acoustic activity that follows each heartbeat to generate time-dependent analog waveforms. The waveforms propagate through shielded, co-axial wires in the cable 14 which connect to the console 10 using a bulkhead connector 12. An analog-to-digital converter in the console 10 converts the analog waveforms to digital ones, which the microprocessor analyzes to determine the patient's blood pressure.

FIGS. 3A and 3B show graphs 50, 52 of the time-dependent electrical waveform 60, optical waveform 62, and acoustic waveform 64 in more detail. Each waveform 60, 62, 64 includes time-dependent features that repeat with each heartbeat. For example, the electrical waveform 60 looks similar to a conventional ECG and features a QRS complex featuring a sharp spike that indicates an initial depolarization of the ventricle. Because of its well-defined features, the QRS complex is relatively easy to detect with a computational algorithm, and serves as an effective ‘marker’ that indicates each individual heartbeat. The optical waveform 62 is measured from underlying capillaries in the patient's chest and features a slowly varying pulse that indicates an increase in volume in the capillaries caused by a propagating pressure wave. Finally, the acoustic waveform features two ‘beats’, each representing a collection of acoustic frequencies, that occur with each heartbeat. The first and second beats represent the sounds made following closure of, respectively, the heart's mitral and aortic valves; these are the conventional ‘lub’ and ‘dub’ heard through a stethoscope.

FIG. 3B graphs a portion of the waveforms highlighted by a box 66 of FIG. 3A, and indicates how a microprocessor preferably analyzes the various features of the electrical waveform 60′, optical waveform 62′, and acoustic waveform 64′ to determine a variety of systolic time intervals. These systolic time intervals are then further processed to determine a patient's real-time blood pressure. As described above, the QRS complex in the electrical waveform 60′, which is caused by initial depolarization of the heart muscle, serves as a marker indicating the start of each heart beat. At a later time, the mitral valve opens and blood flows from the heart's left atrium into the left ventricle. The mitral valve then closes, causing the first beat in the acoustic waveform 64′, and the aortic valve opens shortly thereafter. The opening of the aortic valve does not result in a feature in the acoustic waveform 64′ (only closing valves do this), but is assumed to follow within approximately 10 milliseconds after the closing of the mitral valve. The time difference between the onset of the QRS complex and the opening of the aortic valve is called the ‘pre-injection period’, or PEP. Since the technique described herein does not explicitly measure the opening of the aortic valve, but rather the closure of the mitral valve, it is labeled PEP*. Once the aortic valve opens, the heart pumps a bolus of blood through the aorta, resulting in a pressure wave that propagates through the patient's arterial system. The propagation time of the pressure wave is a strong function of the patient's blood pressure, along with their vascular compliance and resistance. When the pressure wave reaches capillaries in the patient's chest, the rise in pressure causes the capillaries to increase in volume with blood, which in turn increases the amount of optical radiation from the LED of the optical sensor 20 that the flowing blood absorbs. The photodetector in the optical sensor 20 detects this as a time-dependent pulse characterized by a relatively sharp rise time and a slower decay, as indicated by the optical waveform 62′. The time difference between the estimated opening of the aortic valve and the onset of the pulse's rise time is the ‘vascular transit time’ (VTT*). Typically the VTT* decreases with higher blood pressure. The second beat in the acoustic waveform 64′ represents the closure of the aortic valve, and the time period separating this from the estimated opening of the aortic valve is called the ‘left ventricular ejection period’ (LVET*). Finally, the onset of the QRS complex and the foot of the plethysmograph is the pulse transit time (PTT*). Note that the transit time essentially represents the time from when the heart begins to beat to when the pressure wave appears underneath the optical sensor 20. To reach this point, the vascular pathway that the pressure wave must travel is somewhat complicated: it extends through the aorta, the subclavian artery, a series of smaller arteries proximal to the patient's ribs, and finally through relatively small capillaries attached to these arteries. The collective length of this pathway explains the relatively long PTT* shown in FIGS. 3A and 3B.

Other properties known to correlate to blood pressure can also be measured from the optical waveform 62, electrical waveform 60, and acoustic waveform 64. For example, as described below in Table 1, the rise and fall times of the optical waveform 62 can meet this criterion, and thus these properties can be measured from the optical waveform 62. In addition, in some cases the optical waveform 62 will include a primary and secondary peak, separated by a feature called the ‘dicrotic notch’. The microprocessor can be programmed to take a second derivative of the waveform to determine the ratio of the primary and second peaks, and this property has been shown to correlate to blood pressure. In addition, variability in the patient's heartbeat, as measured from each of the electrical waveform 60, optical waveform 62, and acoustic waveform 64, can indicate variation in the patient's blood pressure, and can also be processed by the microprocessor. Heart rates from these three waveforms can be calculated and averaged together to yield a very accurate measure of the patient's real-time heart rate.

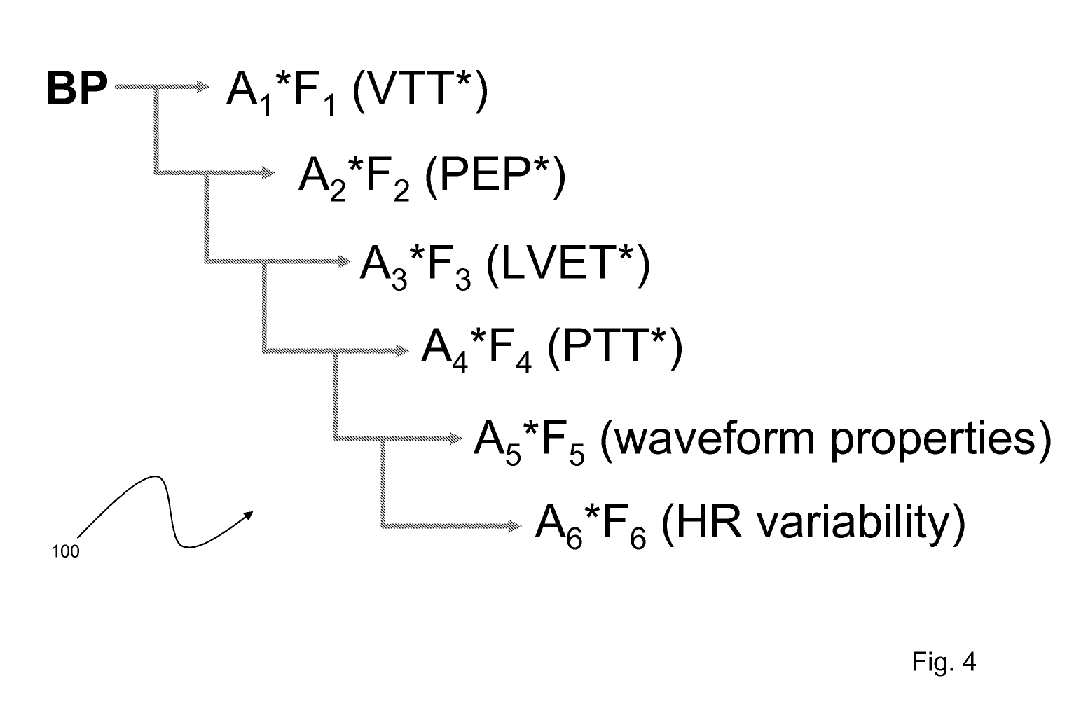

FIG. 4 shows a semi-empirical equation 100 that describes how blood pressure relates to the different time-dependent properties measured by the blood pressure monitor described in FIG. 1. Specifically, during operation, VTT*, PEP*, LVET*, PTT* waveform properties, and heart rate variability can be measured from underneath the patient's sternal notch with the flexible foam pad 16 and processed with the microprocessor in the console 10 to determine the patient's blood pressure. In general, each of these properties, along with other time-dependent waveform properties, has independently been shown to correlate to blood pressure, typically in a linear relationship following an initial calibration. Table 1, below, lists references that describe these properties and the instrumentation used to measure them. The Table is simply meant to list representative documents, and is not meant to be an exhaustive collection of all documents describing a correlation between blood pressure and STIs or time-dependent waveform properties. Each of the references described in Table 1 are hereby incorporated by reference.

TABLE 1
relationship between blood pressure and time-dependent properties measured
from STIs and other waveform properties.

| Property | Reference | Instrumentation |
|---|---|---|
| PTT | U.S. Pats. No. 5,316,008; 5,857,975; | ECG and Pulse Oximeter |
| | 5,865,755; 5,649,543 | |
| VTT | U.S. Pats. No. 6,511,436; 6,599,251; | Paired Optical and Pressure |
| | 6,723,054; 7,029,447 | Sensors; ICG |
| LVET | ‘Short-term variability of pulse pressure | Intra-arterial Catheter |
| | and systolic and diastolic time in heart | |
| | transplant recipients’, Am. J. Physiol. | |
| | Heart Circ. Physiol. 279, H122–H129 | |
| | (2000) | |
| PEP | ‘Relationship between systolic time | Intra-arterial Catheter |
| | intervals and arterial blood pressure’, | |
| | Clin. Cardiol. 9, 545–549, (1986) | |
| PEP/LVET | ‘Systolic Time Intervals in Man’, | Intra-arterial Catheter |
| | Circulation 37, 149–159 (1968) | |
| PPG Width | ‘How does the plethysmograph derived | Pulse Oximeter |
| | from the pulse oximeter relate to arterial | |
| | blood pressure in coronary bypass graft | |
| | patients’, Anesth. Analg. 93, 1466–1471 | |
| | (2001) | |
| PPG Second | ‘Assessment of vasoactive agents and | Pulse Oximeter |
| Derivative | vascular aging by the second derivative of | |
| | the photoplethysmogram waveform’, | |
| | Hypertension 32, 365–370 (1998) | |

FIG. 4 indicates that each of the time-dependent properties correlates with blood pressure according to a function ‘F’ (i.e. F₁, F₂, F₃, F₄, F₅ and F₆), which is typically a linear function characterized by both a slope and y-intercept. The parameter ‘A’ (i.e. A₁, A₂, A₃, A₄, A₅ and A₆) determines the weighting of the function in the blood pressure calculation. Typically the parameters A and F are determined once during an initial calibration period, and then used for all subsequent measurements. For example, during operation the flexible foam pad 16 can be held to the patient's chest, and a button on the console 10 is depressed indicating a calibration is to begin. The pad then measures the time-dependent electrical waveform 60, optical waveform 62, and acoustic waveform 64 and processes them to determine VTT*, PEP*, LVET*, PTT*, any additional waveform properties and heart rate variability. These properties are then stored in non-volatile memory in the console 10. A graphical user interface operating on the console 10 then prompts the user to measure their blood pressure (both systolic and diastolic values) using conventional means, e.g. with a cuff-based device. This can be done at home or in a medical office. The patient then enters the systolic and diastolic values through the graphical user interface and microprocessor stores them in the non-volatile memory. An algorithm operating on the microprocessor performs a simple least-squares fitting routine to determine the slope and y-intercept within each ‘F’ function that relates each property to blood pressure. Weighting parameters ‘A’ are determined prior to any measurements and are loaded into memory during manufacturing.

Once the blood pressure monitor is calibrated, slope and y-intercept values corresponding to each function ‘F’ and weighting factors ‘A’ are stored in memory and are used in subsequent blood pressure calculations along with time-dependent properties measured from the electrical waveform 60, optical waveform 62, and acoustic waveform 64.

In other embodiments the blood pressure monitor 5 is calibrated using pre-set parameters stored in the blood pressure monitor 5 during manufacturing, and is not calibrated using a conventional (e.g. cuff-based) measurement. In this case, for example, clinical studies conducted before manufacturing are used to determine ‘calibrations’ comprising slope, y-intercept, and weighting parameters for specific demographics characterized by biometric parameters such as age, weight, height, gender, and race. After they are determined, these parameters are loaded into non-volatile memory on the monitor during manufacturing. Afterwards, a patient using the blood pressure monitor 5 enters their biometric parameters using the graphical user interface, and an algorithm operating on the monitor analyzes them to determine the appropriate ‘calibration’ to use. The blood pressure monitor 5 uses this ‘calibration’ for all subsequent measurements until the patient enters new biometric parameters.

In another embodiment a ‘universal calibration’, characterized by a single set of slope, y-intercept, and weighting parameters, is determined using clinical studies and stored in non-volatile memory in the blood pressure monitor 5. In this case, the graphical user interface does not include an interface that allows the patient enters biometric or calibration information, and the blood pressure monitor 5 then uses parameters from the ‘universal calibration’ for all subsequent measurements.

In yet another embodiment, the blood pressure monitor 5 may support two or more of the above-mentioned calibration approaches. For example, the blood pressure monitor 5 may have stored in its memory a ‘universal calibration’ and specific ‘calibrations’ characterized by biometric parameters. In addition, the blood pressure monitor 5 may be programmed to accept individual calibrations determined using conventional blood pressure monitors (e.g. cuff-based devices). In this case the graphical user interface is structured so that the patient can easily select the type of calibration to use for each measurement. The patient then proceeds as described above to make each blood pressure measurement.

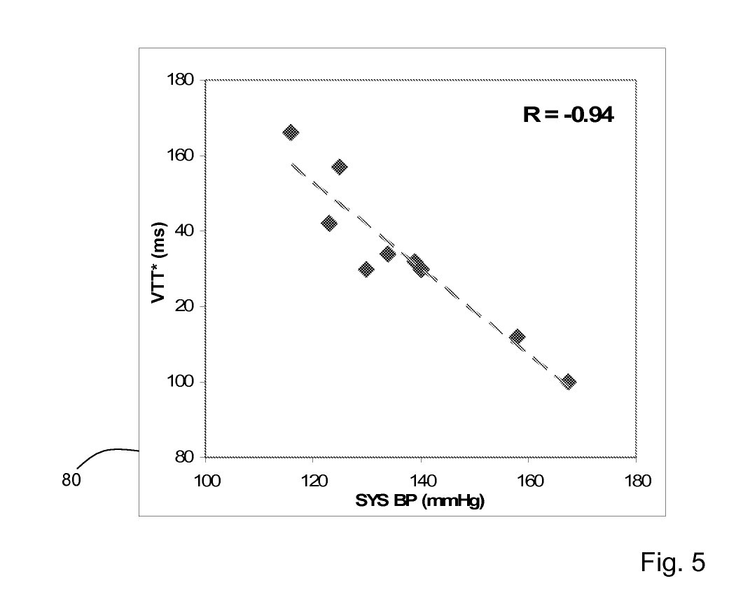

FIG. 5 shows a graph that describes how a systolic time interval measured using the above-described method correlates to a blood pressure measurement from a conventional cuff-based device. Specifically, the graph plots VTT* as a function of systolic blood pressure for a given patient over a range of blood pressures. As described above in FIG. 4, VTT* varies in a linear manner with blood pressure. When fit with a linear function to determine slope and y-intercept for this time-dependent parameter, the fit correlates with the data with an R value of −0.94. This fitting process, for example, could take place during one of the above-described calibration steps.

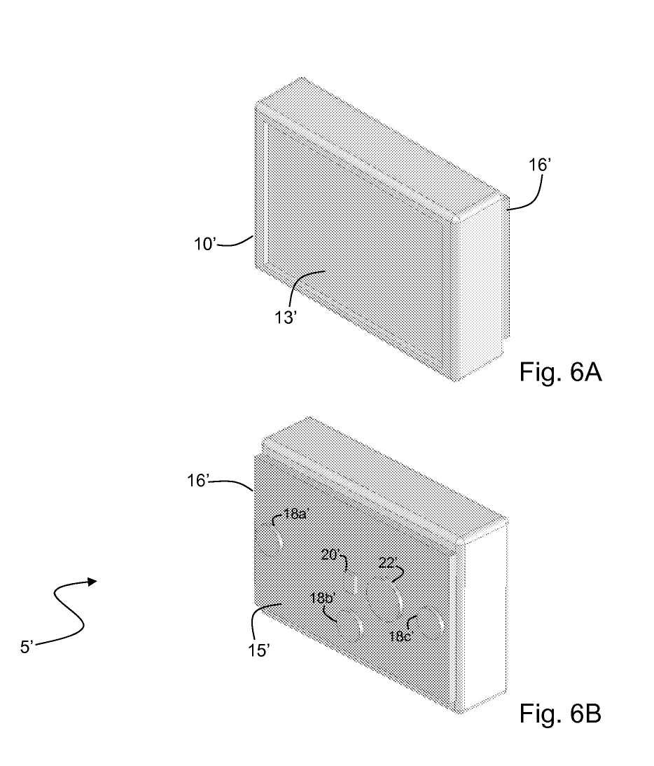

FIGS. 6A and 6B show a blood pressure monitor 5′ corresponding to an alternate embodiment of the invention wherein the flexible foam pad 16′ comprises the substrate 15′ and adheres directly to the back surface of the console 10′. The console 10′ includes a display that operates a touch screen panel and graphical user interface. In this case the flexible foam pad 16′ includes all the sensor elements described with reference to FIG. 1, i.e. two signal and one ground electrodes 18a-c′, an optical sensor 20′ featuring a LED and a photodetector, and a piezoelectric acoustic sensor 22′. In this embodiment the flexible foam pad 16′ connects to power, ground, and signal electrical leads in the console 10′ through a flexible tab connector (not shown in the figure), and there is no cable connecting these two components.

During operation, the patient holds the blood pressure monitor 5′ in one hand and gently pressure the flexible foam pad 16′ to their chest so that the display 13′ faces away from the patient. As with the embodiment shown in FIG. 1, the electrodes 18a-c′, optical 20′ and acoustic 22′ sensors measure, respectively, electrical, optical, and acoustic waveforms similar to those shown in FIGS. 3A and 3B. In this embodiment, the patient cannot clearly see the display 13′, and thus the console includes a piezoelectric ‘beeping’ component (not shown in the figure) that beeps when the measurement is complete. At this point the patient removes the blood pressure monitor 5′ from their chest and views the display 13′ the see the blood pressure reading.

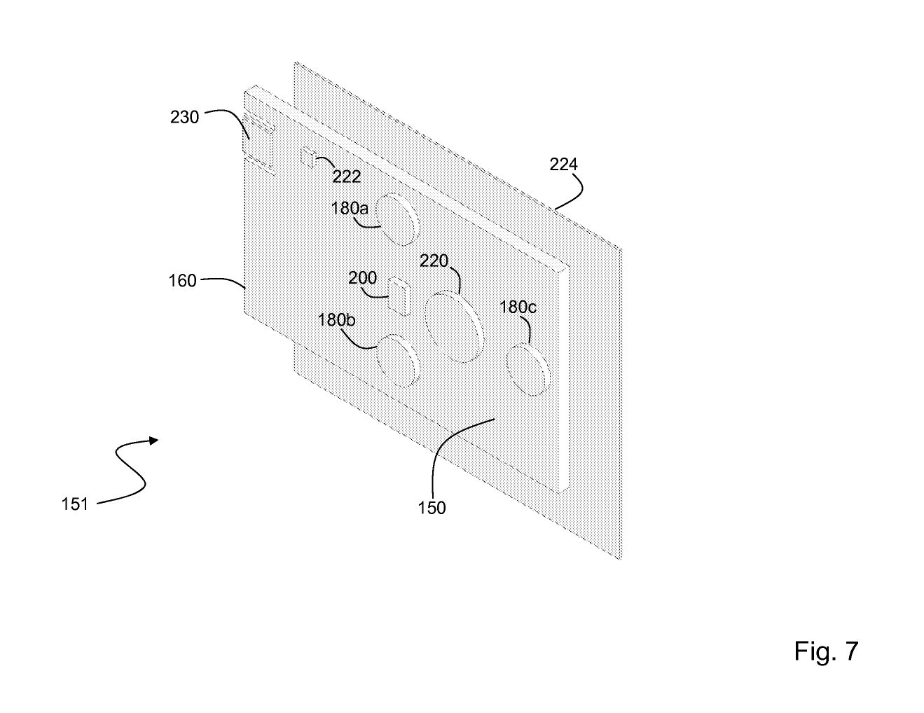

FIG. 7 shows yet another embodiment of the invention wherein an adhesive sensor 151 includes a substrate 150 embedded within a flexible foam pad 160. The foam pad 160 attaches to an adhesive backing 224, allowing the system to be temporarily attached to a patient. In this case, the flexible foam pad 160 includes a tab connector 230 that attaches to a detachable cable (not shown in the figure) that connects to a body-worn console (also not shown in the figure). The flexible foam pad 160 also includes a microchip 222 that stores a serial number in a small-scale, non-volatile memory. During a measurement, the body-worn console connects to the microchip 222 through the tab connector 230 and detachable cable to read the serial number that identifies a particular foam pad 160. The body-worn console also includes a user interface wherein a user (e.g., a medical professional, such as a nurse, or the patient) can enter information describing, e.g., the patient. Software running of a microprocessor in the console associates the serial number to the patient's information.

As described with reference to FIGS. 1, 6A, and 6B, the flexible foam pad 160 includes three electrodes 180a-c, an optical sensor 200, and an acoustic sensor 220. The sensors measure electrical, optical, and acoustic information as describe above to determine the patient's blood pressure.

The flexible foam pad 160 described in FIG. 7 can be used to make quasi-continuous measurements from a patient over an extended period of time (e.g., from several hours to several days). In this case, the adhesive backing 224 attaches to the patient so the three electrodes 180a-c, optical sensor 200, and acoustic sensor 220 contact the patient's chest. One end of the detachable cable connects to the tab connector 230, while the other end attaches to the body-worn console. While the cable is attached, the sensors measure electrical, optical, and acoustic waveforms as described above to determine the patient's blood pressure. During periods where it is not necessary to monitor the patient, the detachable cable detaches from the tab connector 230 while the adhesive backing 224 and flexible foam pad stays adhered 160 to the patient. When the detachable cable is reconnected to the tab connector, the console reads a serial number from the microchip 222 to identify the adhesive sensor 150 as well as the patient it is attached to. Once this is complete, the console continues to measure the patient's blood pressure as described above.

Other embodiments are also within the scope of the invention. For example, the console may include wireless systems (e.g. a wireless modem) or serial port (e.g. a USB port) to connect to an Internet-accessible website. Such systems, for example, are described in the below-mentioned references, the entire contents of which are incorporated herein by reference. In other embodiments, short-range wireless systems connect the flexible foam pad and its associated sensors to the console, making the cable unnecessary. In this case, the flexible foam pad and console have matched wireless transceivers and batteries to power them.

In other embodiments, the optical, electrical, and acoustic sensors are included in a circuit board attached to the end of the cable. In this case the circuit board can ‘snap’ into a disposable adhesive sensor, which in turn attaches to the patient. The disposable adhesive sensor typically includes openings for the optical and acoustic sensors so they can contact the patient to measure, respectively, optical and acoustic signals, as well as Ag/AgCl electrodes covered by a solid gel to measure electrical signals. By including the relatively expensive electrical components in the cable, this embodiment minimizes the cost of the disposable component, which is comprised mostly of an adhesive pad and the electrode materials.

In other embodiments, the flexible foam pad can include optical, electrical, and acoustic sensors on one side, and a finger-clip sensor that includes optical and electrical sensors on the opposing side. In this case, during operation, a patient slides their finger into the finger clip sensor to measure optical and electrical signals from one hand. The patient then simultaneously presses the foam pad against their chest so that the optical, electrical, and acoustic sensors measure their respective signals as described above. A cable connecting the flexible foam pad to the console transmits the signals from the patient's hand and chest to the microprocessor, which then processes them as described above to determine systolic time intervals, and particularly PTT, to determine blood pressure.

In other embodiments, the optical, electrical, and acoustic waveforms can be processed to determine other vital signs. For example, relatively low-frequency components of an ‘envelope’ describing both the electrical and optical waveforms can be processed to determine respiratory rate. This can be done, for example, using an analysis technique based on Fourier Transforms. In other embodiments, the substrate can be modified to include light sources (e.g. LEDs) operating in both the red (e.g. λ=600-700 nm) and infrared (λ=800-900 nm) spectral regions. With these modifications, using techniques know in the art, that substrate can potentially measure pulse oximetry in a reflection-mode configuration. In still other embodiments, time-dependent features from the PCG can be analyzed to determine cardiac properties such as heart murmurs, lung sounds, and abnormalities in the patient's mitral and aortic valves.

In other embodiments, the blood pressure monitor can connect to an Internet-accessible website to download content, e.g. calibrations, text messages, and information describing blood pressure medication, from an associated website. As described above, the monitor can connect to the website using both wired (e.g. USB port) or wireless (e.g. short or long-range wireless transceivers) means.

In addition to those described above, a number of methods can be used to calculate blood pressure from the optical, electrical, and acoustic waveforms. These are described in the following co-pending patent applications, the contents of which are incorporated herein by reference: 1) CUFFLESS BLOOD-PRESSURE MONITOR AND ACCOMPANYING WIRELESS, INTERNET-BASED SYSTEM (U.S. Ser. No. 10/709,01; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) VITAL SIGN MONITOR FOR ATHLETIC APPLICATIONS (U.S. Ser. No.; filed Sep. 13, 2004); 5) CUFFLESS BLOOD PRESSURE MONITOR AND ACCOMPANYING WIRELESS MOBILE DEVICE (U.S. Ser. No. 10/967,511; filed Oct. 18, 2004); and 6) BLOOD PRESSURE MONITORING DEVICE FEATURING A CALIBRATION-BASED ANALYSIS (U.S. Ser. No. 10/967,610; filed Oct. 18, 2004); 7) PERSONAL COMPUTER-BASED VITAL SIGN MONITOR (U.S. Ser. No. 10/906,342; filed Feb. 15, 2005); 8) PATCH SENSOR FOR MEASURING BLOOD PRESSURE WITHOUT A CUFF (U.S. Ser. No. 10/906,315; filed Feb. 14, 2005) 9) PATCH SENSOR FOR MEASURING VITAL SIGNS (U.S. Ser. No. 11/160,957; filed Jul. 18, 2005); 10) WIRELESS, INTERNET-BASED SYSTEM FOR MEASURING VITAL SIGNS FROM A PLURALITY OF PATIENTS IN A HOSPITAL OR MEDICAL CLINIC (U.S. Ser. No. 11/162,719; filed Sep. 9, 2005); 11) HAND-HELD MONITOR FOR MEASURING VITAL SIGNS (U.S. Ser. No. 11/162,742; filed Sep. 21, 2005); 12) CHEST STRAP FOR MEASURING VITAL SIGNS (U.S. Ser. No. 11/306,243; filed Dec. 20, 2005); 13) SYSTEM FOR MEASURING VITAL SIGNS USING AN OPTICAL MODULE FEATURING A GREEN LIGHT SOURCE (U.S. Ser. No. 11/307,375; filed Feb. 3, 2006); 14) BILATERAL DEVICE, SYSTEM AND METHOD FOR MONITORING VITAL SIGNS (U.S. Ser. No. 11/420,281; filed May 25, 2006); and 15) SYSTEM FOR MEASURING VITAL SIGNS USING BILATERAL PULSE TRANSIT TIME (U.S. Ser. No. 11/420,652; filed May 26, 2006).

Still other embodiments are within the scope of the following claims.

We claim as our invention:

1. A method for measuring a blood pressure value of a user, the method comprising:
generating an optical waveform from a first signal detected by an optical sensor comprised by a substrate in contact with a user;
generating an electrical waveform from a second signal detected by at least one electrode comprised by the substrate in contact with a user;
generating an acoustic waveform from a third signal detected by an acoustic sensor comprised by the substrate in contact with a user;
determining at least one time-dependent parameter by analyzing at least the optical waveform and the acoustic waveform; and,
processing the at least one time-dependent parameter to determine the blood pressure value for the user.

2. The method of claim 1, wherein generating the optical waveform further comprises irradiating a first region of the user with a light source and detecting radiation reflected from the first region with a photodetector.

3. The method of claim 2, further comprising digitizing a signal from the photodetector with an analog-to-digital converter.

4. The method of claim 2, further comprising irradiating a first region of the user with radiation having a wavelength between 520 and 590 nm.

5. The method of claim 2, further comprising irradiating a first region of the user with radiation having a wavelength between 800 and 1100 nm.

6. The method of claim 1, wherein generating the electrical waveform further comprises detecting a first electrical signal with a first electrode comprised by the substrate, detecting a second electrical signal with a second electrode, and electrically processing the first and second electrical signals.

7. The method of claim 6, wherein the electrically processing further comprises differential amplifying the first and second electrical signals with an amplifier system.

8. The method of claim 6, further comprising detecting a third electrical signal with a third electrode.

9. The method of claim 8, further comprising detecting a ground signal with a ground electrode.

10. The method of claim 1, wherein generating the acoustic waveform comprises detecting a time-dependent signal representing sounds from the user's heart.

11. The method of claim 1, wherein generating the acoustic waveform comprises detecting a time-dependent signal with a piezoelectric acoustic sensor comprised by the substrate.

12. The method of claim 11, wherein generating the acoustic waveform comprises detecting a time-dependent signal with a piezoelectric acoustic sensor in contact with an impedance-matching gel.

13. The method of claim 10, wherein generating the acoustic waveform comprises detecting a phonocardiogram from the user.

14. The method of claim 10, wherein generating the acoustic waveform comprises detecting at least one heart sound from the user.

15. The method of claim 1, further comprising determining the at least one parameter by analyzing a first point in time from a pulse comprised by the optical waveform and a second point in time from at least one feature representing a heart sound comprised by the acoustic waveform.

16. The method of claim 1, further comprising determining a second parameter by analyzing a third point in time from a QRS complex comprised by the electrical waveform and a fourth point in time from a pulse comprised by the optical waveform.

17. The method of claim 1, further comprising processing the at least one parameter with a mathematical model to determine the blood pressure value for the user.

18. The method of claim 17, further comprising processing the at least one parameter with a linear model characterized by a slope and a y-intercept to determine the blood pressure value for the user.

19. The method of claim 18, further comprising processing the at least one parameter with calibration information to determine a blood pressure value for the user.

20. A method for measuring a real-time blood pressure value for a user, the method comprising:
placing a device in proximity to a sternal notch of the user, the device comprising an optical sensor, an acoustical sensor, a plurality of electrodes and a processor;
generating an optical waveform from a signal from the optical sensor, an acoustical waveform from the acoustical sensor and an electrical waveform from the plurality of electrodes;
processing the optical waveform, the acoustical waveform and the electrical waveform to determine a plurality of values comprising a PTT value, a PEP value, a VTT value and a LVET value; and
generating a real-time blood pressure value for the user from at least one of the plurality of values.

21. A method for measuring a blood pressure of a user, the method comprising:
generating an optical waveform from a first signal detected by a first optical sensor comprised by a substrate in contact with a user, the optical waveform comprising a time-dependent pulse;
generating an electrical waveform from a second signal detected by at least one electrode comprised by the substrate in contact with a user, the electrical waveform comprising a QRS complex;
generating an acoustic waveform from a third signal detected by an acoustic sensor comprised by the substrate in contact with a user, the acoustic waveform comprising at least one feature representing a heart sound;
determining a first parameter by analyzing a first point in time from the pulse comprised by the optical waveform and a second point in time from the at least one feature representing a heart sound comprised by the acoustic waveform;
determining a second parameter by analyzing a third point in time from the QRS complex comprised by the electrical waveform at least one point in time comprised by one of the acoustic waveform and the optical waveform; and
processing both the first and second parameters to determine a blood pressure value for the user.

22. A device for measuring a blood pressure value of a user, the device comprising:
a substrate;
an optical sensor, comprised by the substrate, comprising a light source and a photodetector configured to generate an optical waveform from a first signal detected after the optical sensor irradiates the user;
an electrical sensor, comprised by the substrate, comprising at least one electrode and configured to generate an electrical waveform from a second signal when the at least one electrode contacts the user;
an acoustic sensor, comprised by the substrate, configured to generate an acoustic waveform from a third signal when the acoustic sensor is proximal to the user; and,
a processor, in electrical contact with the optical, electrical, and acoustic sensors, and configured to receive the optical, electrical, and acoustic waveforms, the processor further configured to determine at least one parameter by analyzing the optical waveform and the acoustic waveform and to process the at least one parameter to determine the blood pressure value for the user.

23. The device of claim 22, wherein the substrate comprises a printed circuit board.

24. The device of claim 22, wherein the substrate comprises a flexible material.

25. The device of claim 22, wherein the optical sensor further comprises a light source configured to emit radiation having a wavelength between 520 and 590 nm.

26. The device of claim 22, wherein the optical sensor further comprises a light source configured to emit radiation having a wavelength between 800 and 1100 nm.

27. The device of claim 22, wherein the electrical sensor further comprises at least two electrodes.

28. The device of claim 27, wherein the at least two electrodes are disposed on opposite sides of the substrate.

29. The device of claim 27, wherein the at least two electrodes are separated by a distance of at least two inches.

30. The device of claim 22, wherein the at least one electrode further comprises an Ag/AgCl material.

31. The device of claim 30, wherein the at least one electrode further comprises a conductive gel.

32. The device of claim 31, wherein on a first surface the conductive gel contacts the Ag/AgCl material, and on a second surface the conductive gel contacts a protective layer.

33. The device of claim 22, wherein the acoustic sensor further comprises a microphone.

34. The device of claim 33, wherein the microphone further comprises a piezoelectric material.

35. The device of claim 22, further comprising an analog-to-digital converter in electrical contact with the optical, electrical, and acoustic sensors and the processor.

36. The device of claim 35, wherein the analog-to-digital converter is configured to receive an analog optical waveform from the optical sensor, an analog electrical waveform from the electrical sensor, and an analog acoustic waveform from the acoustic sensor, the analog-to-digital converter further configured to digitize the analog optical, electrical, and acoustic waveforms to generate digital optical, electrical, and acoustic waveforms.

37. The device of claim 36, wherein microprocessor is configured to receive the digital optical, electrical, and acoustic waveforms from the analog-to-digital converter.

38. A device for measuring a blood pressure value of a user, the device comprising:
an adhesive backing;
a substrate, attached to the adhesive backing, comprising;
an optical sensor comprising a light source and a photodetector configured to generate an optical waveform from a first signal detected when the optical sensor irradiates the user;
an electrical sensor comprising at least one electrode and configured to generate an electrical waveform from a second signal when the at least one electrode contacts the user; and
an acoustic sensor configured to generate an acoustic waveform from a third signal when the acoustic sensor is proximal to the user; and,
a connector, attached to the substrate and in electrical contact with the optical, electrical, and acoustic sensors; and
a processor, in electrical contact with the connector, and configured to receive the optical, electrical, and acoustic waveforms, the processor further configured to determine at least one parameter by analyzing the optical waveform and the acoustic waveform and to process the at least one parameter to determine the blood pressure value for the user.

39. A device for measuring a blood pressure value of a user, the device comprising:
a substrate comprising:
an optical sensor comprising a light source and a photodetector configured to generate an optical waveform from a first signal measured when the optical sensor irradiates the user;
an electrical sensor comprising least two electrodes and configured to generate an electrical waveform from a second signal when the at least two electrodes contact the user;
an acoustic sensor configured to generate an acoustic waveform from a third signal when the acoustic sensor is proximal to the user; and
a console, attached directly to the substrate, comprising:
a display; and
a processor configured to receive the optical, electrical, and acoustic waveforms, the processor further configured to determine at least one parameter by analyzing the optical waveform and the acoustic waveform and to process the at least one parameter to determine the blood pressure value for the user.

40. A device for measuring a blood pressure value of a user, the device comprising:
a substrate comprising:
an optical sensor comprising a light source and a photodetector configured to generate an optical waveform from a first signal detected when the optical sensor irradiates the user;
an electrical sensor comprising at least one electrode and configured to generate an electrical waveform from a second signal when the at least one electrode contacts the user;
an acoustic sensor configured to generate an acoustic waveform from a third signal when the acoustic sensor is proximal to the user;
a cable in electrical contact with the substrate; and
a console, in electrical contact with the cable, comprising:
a display comprising a touch panel; and
a processor configured to receive the optical, electrical, and acoustic waveforms, the processor further configured to determine at least one parameter by analyzing the optical waveform and the acoustic waveform and to process the at least one parameter to determine the blood pressure value for the user.
